(19) United States
(12) Patent Application Publication
(10) Pub. No.: US 2024/0165291 A1
(43) Pub. Date: May 23, 2024

(54) SUPERABSORBENT CHITOSAN WOUND DRESSINGS, MANUFACTURING METHODS AND APPLICATIONS THEREOF

(71) Applicant: ADVAMEDICA INC., Cambridge, MA (US)

(72) Inventors: Leo MAVELY, Ahmedabad (IN); Kiran SONAJE, Ahmedabad (IN); Animesh AGRAWAL, Ahmedabad (IN); Pavan KUMAR PALAMKOTA, Ahmedabad (IN); Venkat RATNAM DEVADASU, Ahmedabad (IN); Anup KUMAR, Ahmedabad (IN)

(21) Appl. No.: 18/552,129

(22) PCT Filed: Mar. 25, 2022
(86) PCT No.: PCT/IB2022/052749

§ 371 Date: Sep. 22, 2023

(30) Foreign Application Priority Data
Mar. 26, 2021 (IN) 202141013261

(51) Int. Cl. A61L 15/40; A61F 13/00; A61L 15/60; C08B 37/08; C08L 5/08

(52) U.S. Cl. CPC A61L 15/40; A61F 13/00063; A61F 13/00995; A61L 15/60; C08B 37/003; C08L 5/08; C08L 2203/02; C08L 2205/025

(57) ABSTRACT

The present disclosure provides a superabsorbent dressing comprising non-woven protonated chitosan fabric/sheet that shows superior fluid absorption and retention capacity, enhanced tensile strength and coherency. The superabsorbent dressings of the present disclosure comprise chitosan fibers composed of chitosan amide and an acid salt of chitosan. The present disclosure also provides a continuous method and a device for manufacturing the chitosan dressings of the disclosure.

### CROSS-REFERENCE TO RELATED APPLICATIONS

The present application claims the benefit of priority to Indian Provisional Application No. 202141013261, filed on Mar. 26, 2021, the contents of which are hereby incorporated by reference herein in their entirety.

### TECHNICAL FIELD

The present disclosure relates to the field of medical dressings. Particularly, the present disclosure relates to superabsorbent chitosan dressings and methods of preparing them. The chitosan dressings of the present disclosure have superior exudate absorption capacity with excellent wet tensile strength allowing prolonged contact with wound surface without losing integrity. Such dressings help in wound management by achieving hemostasis, absorbing the wound exudates, providing antimicrobial activity, faster wound healing, and symptomatic pain relief.

### BACKGROUND OF THE DISCLOSURE

Chitosan is a derivative of chitin that is obtained from natural sources like the shellfish, insects and mushrooms. Chitosan has been widely investigated for use in wound dressings. Chitosan in its natural form is composed of tightly packed polymer chains containing monomers of poly-N-glucosamine and poly-N-acetyl-glucosamine. The unmodified chitosan is generally not soluble in any physiologically relevant media such as water, saline or buffers. However, on mixing with dilute acids the primary amine groups on chitosan undergo protonation which leads to charge repulsion between adjacent chains followed by fluid absorption, swelling of polymer chains, and subsequent dissolution of chitosan. The protonated amine groups of chitosan also play an important role in many of its biological properties such as muco/bioadhesion, hemostasis, platelet activation and antibacterial properties.

For the above-discussed reasons, protonated form of chitosan is preferred in making chitosan-based wound dressings. Different forms of chitosan have been reported such as gels, powders, films, sponges, chitosan impregnated gauzes, as well as non-woven gauze made using chitosan fibers. Among these, chitosan fiber/gauze dressings are most useful as wound dressings due to their conformability, flexibility, and capacity to be produced on a larger scale as compared to films or sponges. Chitosan fibers are generally produced by the wet spinning method, which involves precipitating the chitosan solution in alkaline solutions (e.g., sodium hydroxide or potassium hydroxide) which neutralizes the cationic charge on chitosan molecules (deprotonation). However, these chitosan fibers are water insoluble and contain neutralized form of chitosan which has poor fluid absorption capacity, does not offer exudate locking properties and lacks sufficient wound healing properties due to the lack of cationic charge.

Therefore, deprotonated/neutral chitosan fibers are generally processed by different methods to convert them into forms suitable for wound management applications. Currently reported methods to convert chitosan fibers into wound dressing involve first converting chitosan fibers into a water-soluble or swellable form of chitosan fibers (modified chitosan fibers) and then making a woven or non-woven gauze using these modified chitosan fibers. The final properties of chitosan dressings prepared in this manner depend on the properties of chitosan fibers. For example, if chitosan fibers are not sufficiently protonated, the final dressing prepared from them does not provide sufficient exudate absorption capacity and does not promote wound healing. On the other hand, if the chitosan fibers are excessively protonated, the resulting chitosan dressing instantly dissolves/disintegrates after contacting the exudate and does not provide prolonged exudate absorption property. Therefore, chitosan dressings that provide excellent exudate absorption capacity and at the same time have superior wet strength are desirable. The superior wet strength allows for their prolonged contact with wound surface without losing integrity, also helps in easy and complete removal of chitosan dressing from the wound surface.

US20150011503A1 discloses a chitosan dressing obtained by mixing partly acid activated fibers and cross-linked chitosan fibers and needle punching these fibers to obtain a non-woven chitosan gauze. US20070237811A1 discloses a non-woven chitosan fabric that is supported with cotton, nylon, polyester, rayon, or the mixtures of these. U.S. Pat. No. 7,141,714B2 discloses that the chitosan fibers can be activated with acid and converted into woven or nonwoven gauze later. EP2695622B1 discloses a manufacturing process for a wound dressing composed of acylated chitosan fibers. For the acylation process, chitosan fibers were submersed in a reaction solution containing succinic anhydride and ethanol followed by drying through heating at different temperatures for different times. The reaction solution was squeezed off and the fibers were washed with ethanol or ethanol/tween mixture to remove the residual substances. In this manufacturing process, acylated chitosan fibers or standard chitosan fibers were converted into gauze dressings through knitting, weaving or via any non-woven processing methods. In this, acylated chitosan based wound dressing showed a high wet strength and a higher absorption capacity. US20110311632A1 discloses curing of lyophilized chitosan at higher temperature to increase the strength of the dressing. Some studies also propose using chitosan powder for wound management. The fibers form a cohesive structure whereas powder has no such ability.

All these prior arts disclose preparation of chitosan gauze dressings using chitosan powder or chitosan fibers as starting materials and use of chemical crosslinking or derivatization of chitosan to its water-soluble forms such as acylated chitosan or carboxymethyl chitosan or acid treatment of chitosan fibers prior to its conversion into woven or non-woven gauze. Overall, the methods described in prior art are multi-step methods, which are time consuming, expensive and cumbersome. Additionally, in some cases where acidified chitosan fibers are mixed with crosslinked fibers, getting a uniform distribution of mixed fibers throughout the final dressings is practically impossible. Such dressings tend to have some regions which swell more due to higher content of acidified chitosan than others which do not swell at all due to presence of only crosslinked chitosan fibers. Further, fluid absorbency and retention capacity of dressings prepared from acidified chitosan fibers are still relatively lower.

Therefore, there is a need for a chitosan wound dressing that can form a uniform cohesive gel and interlock the wound exudate. Further a large-scale manufacturing method is needed that gives final dressing with uniform swelling properties.

The present invention attempts to address this need by providing superabsorbent chitosan wound dressings which do not use any chemical crosslinking agents and are obtained by a continuous method that converts unmodified chitosan non-woven fabric into superabsorbent dressings with inherent fortified structure in an economical and rapid manner.

### Statement of the Disclosure

The present disclosure provides a superabsorbent dressing comprising chitosan fibers, wherein the chitosan fibers comprise an acid salt of chitosan (protonated chitosan) in the core and the chitosan amide on the surface of the fibers. The protonated chitosan in the fiber core absorbs fluids, swells, and converts the fibers into a gel. While the outer layer of chitosan amide prevents dissolution of the swollen fibers thereby strengthening the gelled dressing.

In some embodiments, the chitosan amide constitutes about 5-20% by weight of the dressing. The present disclosure also provides a superabsorbent dressing comprising chitosan fibers prepared by a method comprising: a) dipping a fabric/sheet/roll of neutral chitosan fibers in a treatment solution comprising an acid and an alcohol and optionally a surfactant to obtain a protonated chitosan fabric/sheet/roll; b) removing excess amount of said treatment solution from the protonated chitosan fabric/sheet/roll while maintaining chitosan to treatment solution ratio of 1:2 to 1:10; and c) exposing the protonated chitosan fabric/sheet/roll at a temperature of about 60° C. or more for controlled amide-fortification of protonated chitosan to obtain the proton-amide fortified chitosan dressings.

The present disclosure further provides a superabsorbent dressing comprising chitosan fibers composed of chitosan amide and an acid salt of chitosan, wherein the dressing exhibits one or more of the following properties: a) a fluid absorbency of about 14, 20, 25, 30, 35, 40, 45, or 50 g/g or more; b) a dry tensile strength of about 15 N or more, 20 N or more or about 30 N or more; c) a wet tensile strength of about 1 N or more, 5 N or more, or 10 N or more; and/or d) a fluid retention capacity of about 40% or more. Each chitosan fiber comprises protonated chitosan (the acid salt of chitosan) in the core and chitosan amide on the surface of the fiber.

The present disclosure provides a method for preparing a superabsorbent chitosan dressing comprising chitosan fibers composed of chitosan amide and an acid salt of chitosan, said method comprising: a) dipping a non-woven unprotonated chitosan fabric/sheet/roll in a treatment solution comprising an acid and an alcohol and optionally a surfactant to obtain a treated chitosan fabric; b) removing excess amount of said treatment solution from the treated chitosan fabric/sheet/roll; and c) exposing the protonated chitosan fabric/sheet/roll at a temperature of about 60° C. or more for controlled amide-fortification of protonated chitosan to obtain the superabsorbent chitosan dressings, wherein the method is a continuous process.

The present disclosure also provides a device for preparing a superabsorbent chitosan dressing comprising proton-amide-fortified chitosan fibers in a continuous manner, said device comprising: an unwinder (101); a dipping chamber (102); squeezer/padding means (103); a drive/belt (104); a drying chamber (105); and a winder (106).

### BRIEF DESCRIPTION OF THE FIGS

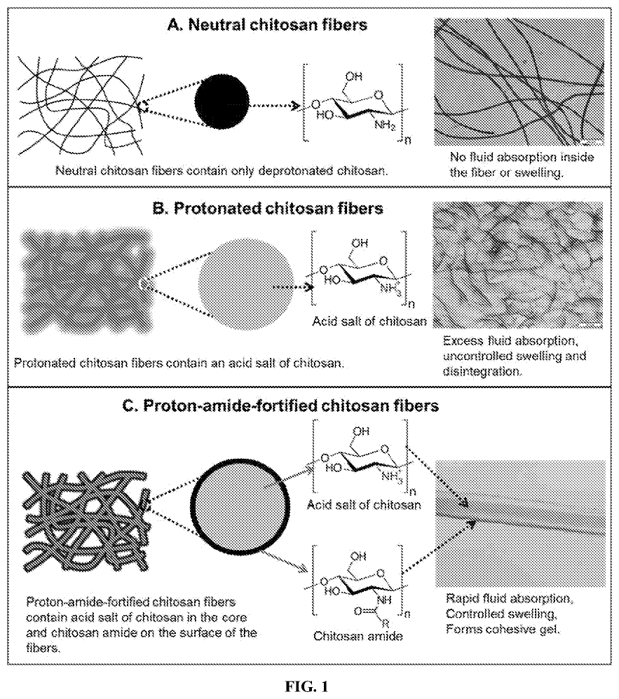

FIG. 1 shows a schematic representation showing the composition, chemical structures and microscopic images of different types of chitosan fibers in presence of water.

Top Panel: The neutral chitosan fibers contain the deprotonated chitosan, which is not soluble in water, hence it does not absorb fluid or swell in the presence of water.

Middle panel: The protonated chitosan fibers contain an acid salt of chitosan, which is able to absorb the excess water and swell to an extent that it starts to disintegrate.

Bottom Panel: The superabsorbent-fortified chitosan fibers of present disclosure contain acid-salt of chitosan in the core and chitosan amide on the surface. The acid salt of chitosan is water soluble and rapidly absorbs the fluids inside the fiber. While chitosan amide is water insoluble, which prevents the disintegration of the swelled fibers giving a fortified cohesive gel after fluid absorption.

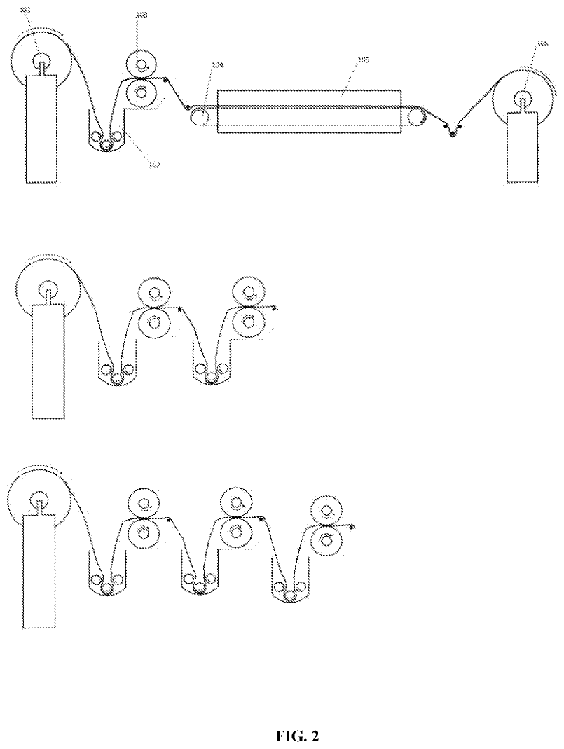

FIG. 2 shows a schematic of an exemplary set-up that can be used in the continuous method of chitosan fabric/sheet/roll treatment. The set-up contains unwinder (101), reagent container (102), squeezer (103), drive/belt (104), drying chamber (105) and winder (106). The modifications of the set-up are possible to accommodate the reagents by increasing the number of reagent containers and squeezers.

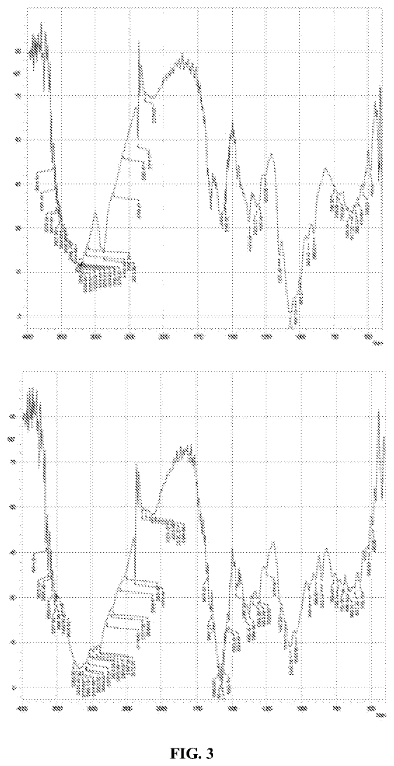

FIG. 3 shows the FTIR spectra indicating the presence of lactamide in the dressings. The upper panel shows the FTIR spectra of untreated/unprotonated chitosan fabric and the bottom panel shows the FTIR spectra of the superabsorbent chitosan dressing of present disclosure.

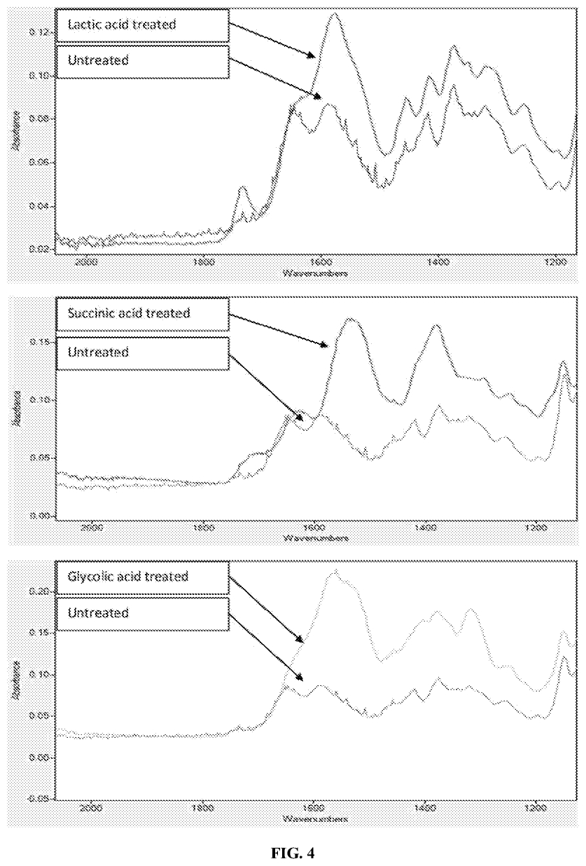

FIG. 4 shows the FTIR spectra of the treated and untreated chitosan fabric. The peaks corresponding to lactamide can be observed at 1735 cm⁻¹ and 1580 cm⁻¹ (Top), the peak corresponding to succinimide can be observed at 1540 cm⁻¹ (Middle) and the peak corresponding to glycolic acid amide can be observed at 1560 cm⁻¹ when compared with the untreated fabric (Bottom).

FIG. 5 shows a light microscopic image of fibers from untreated fabric showing the fiber diameter around 15 μm.

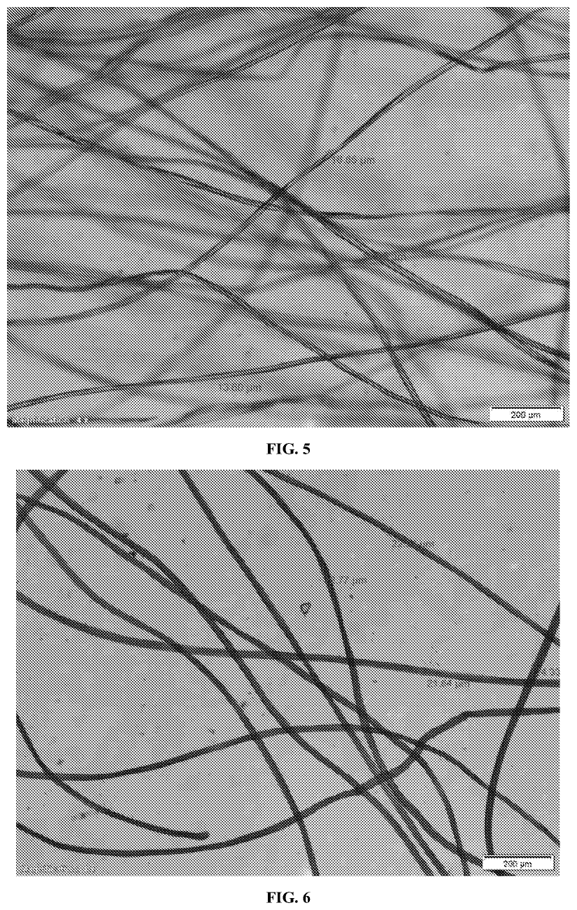

FIG. 6 shows a light microscopic image of fibers from untreated fabric when contacted with a colored aqueous solution showing the fiber diameters of around 20 μm.

FIG. 7 shows a light microscopic image of complete dissolution of protonated fibers obtained after treating chitosan fabric with a treatment solution and drying at a lower temperature (25° C.). When contacted with a colored aqueous solution, the fibers start gelling and dissolve immediately as individual fibers are not visible in this image.

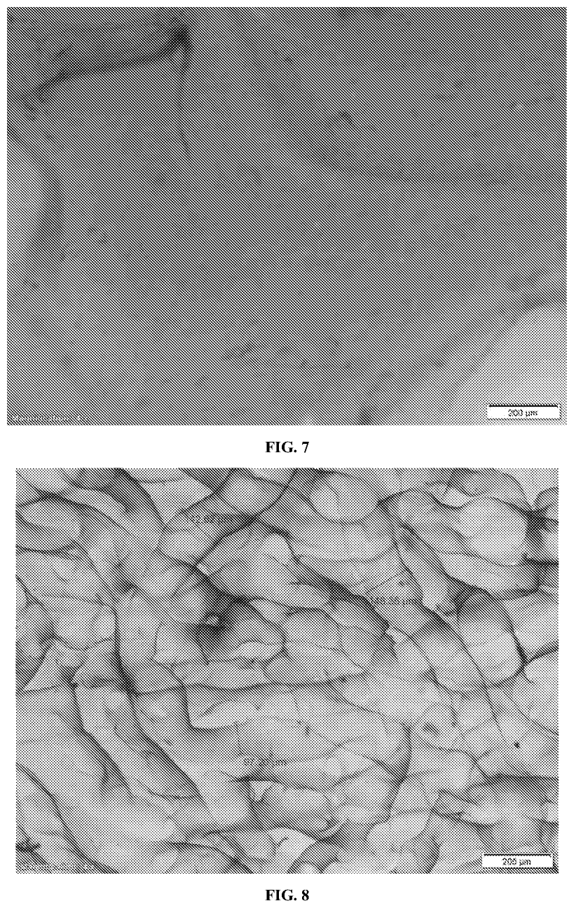

FIG. 8 shows a light microscopic image of fibers from chitosan fabric treated with a treatment solution at a suboptimum temperature (60° C.) when contacted with a colored aqueous solution showing the fiber diameters of around 100 μm. It can be observed clearly that the fibers are swollen but they have burst due to excessive swelling and lack of amide fortification on fiber surface.

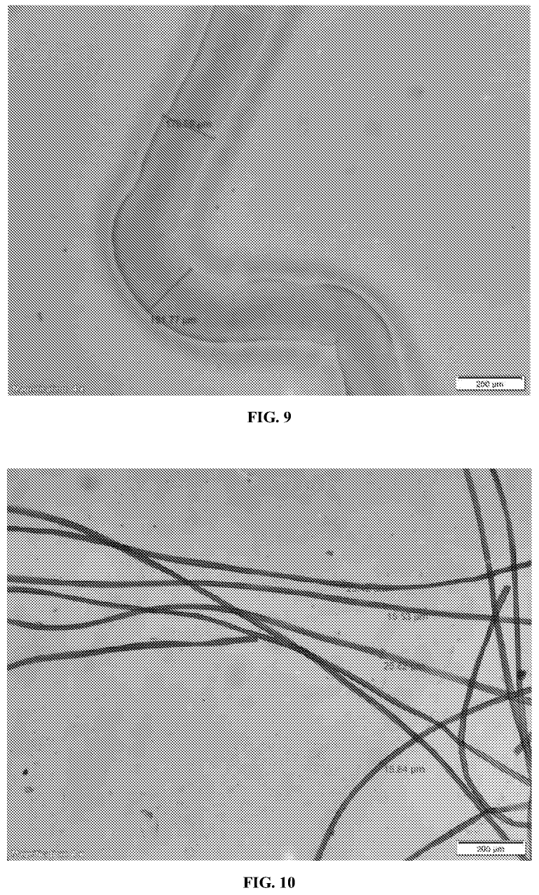

FIG. 9 shows a light microscopic image of fibers from chitosan fabric treated with a treatment solution at an optimum temperature (80° C.-110° C.) when contacted with a colored aqueous solution showing the fiber diameters of around 180 μm. It can be observed clearly that the fibers are swollen and covert into cohesive gel with intact surface structure holding liquid within it. This can be attributed to the proper surface modification with the amide.

FIG. 10 shows a light microscopic image of fibers from fabric treated with a treatment solution at a higher temperature (120° C.), when contacted with a colored aqueous solution showing the fiber diameters of around 20 μm. It can be observed clearly that the fibers do not absorb water or swell, which can be attributed to their impermeable surface due to higher chitosan amide content in the fibers.

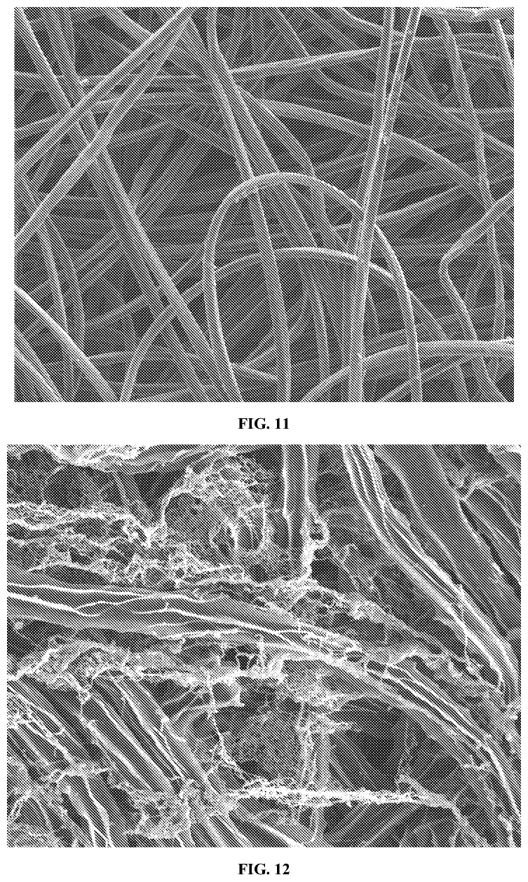

FIG. 11 shows a scanning electron microscopic image of the fabric without any treatment.

FIG. 12 shows a scanning electron microscopic image showing the insoluble chitosan amide layer on the fibers. To take these images, the treated fabric was first dipped in water and subjected to snap freezing in liquid nitrogen. Then the fabric was freeze dried and scanning microscopic image was taken. The porous mesh like structures representing the acidified chitosan core, and the intact smooth structures representing the chitosan amide surface of the proton-amide-fortified chitosan fibers are clearly visible.

FIG. 13 shows the absorbency behavior of the superabsorbent chitosan dressing of the present disclosure (right image and a marketed product (left image). The uniformity in fluid absorbency is clearly visible in the dressings (right image) of present disclosure in comparison to the acidified chitosan dressings (left image).

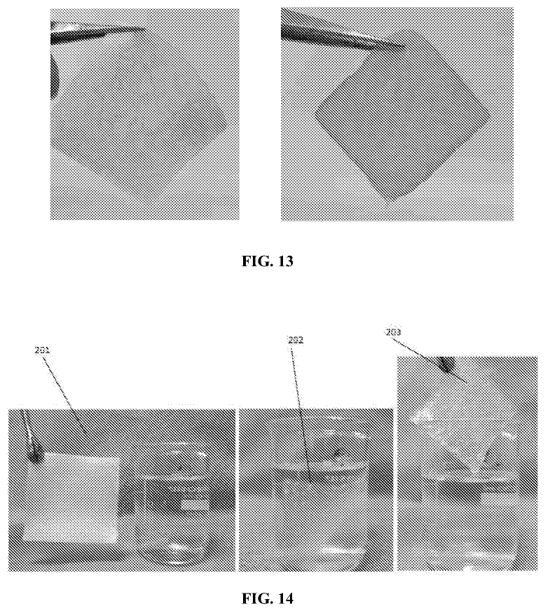

FIG. 14 shows the cohesive structure of the chitosan dressing of the present disclosure before (201), during (202) and after the standard dressing integrity test (203).

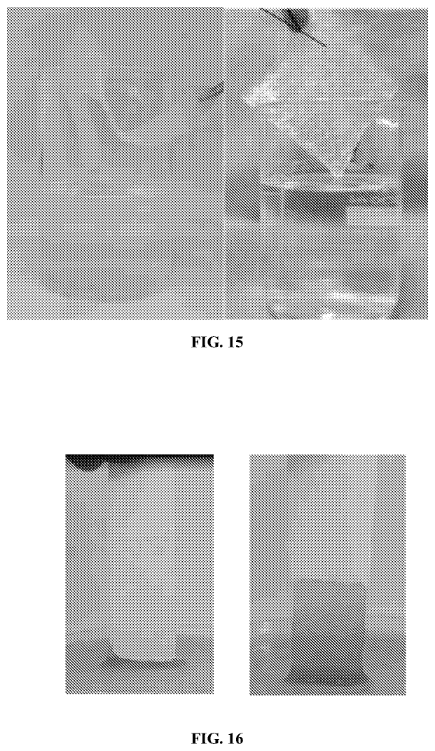

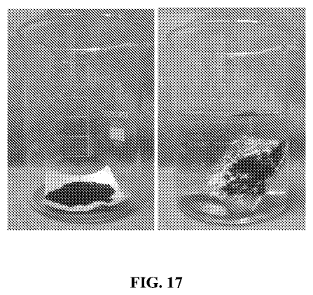

FIG. 15 shows the cohesive structure of the chitosan dressing of the present disclosure (right image) and the marketed product (left image). The dressing of the present disclosure (right image) shows good cohesion after moisture absorption whereas the marketed product (left image) lost its structure and easily disintegrates after moisture absorption.

FIG. 16 shows the fluid uptake in 20 seconds. Acidified chitosan dressing (left image) takes up very little fluid than the chitosan dressing (right image) of the present disclosure.

FIG. 17 shows the hemostatic efficacy of the chitosan dressing of the present disclosure. The dressing rapidly absorbs the blood(left image) and does not leak any free blood after adding the water (right image) indicating that a strong blood clot is formed within the dressing.

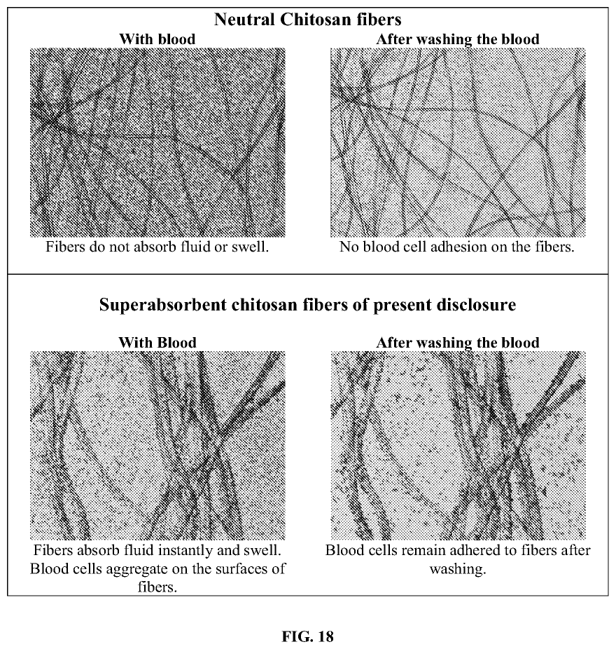

FIG. 18 shows the adhesion and aggregation of blood cells on the different types of chitosan fibers. The neutral chitosan fibers (top row) do not show any fluid absorption or swelling in presence of blood (Top panel, left image). Additionally, no blood cell adhesion was observed when the sample was washed with saline to remove non-adherent cells (top panel right image). On the other hand, the super absorbent fibers of present disclosure (bottom row) showed rapid absorption and swelling in presence of blood and cells could be seen aggregating at the fiber surfaces (bottom panel, left image). When the samples were washed with saline, the blood cells remain adhered to the swollen fibers (bottom panel, right image), indicating a strong charge-based adhesion between blood cells and the superabsorbent chitosan fibers of the present disclosure.

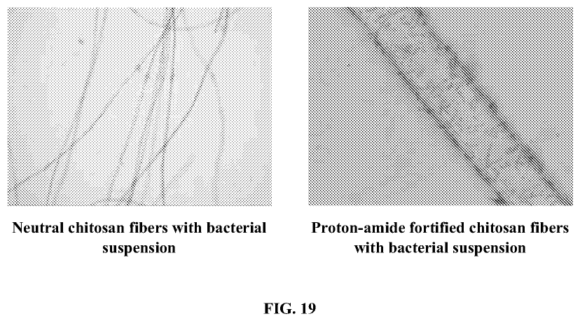

FIG. 19 shows the sequestration of bacterial cells by different types of chitosan fibers. The neutral chitosan fibers (left) do not show any attachment of bacteria, but the super absorbent fibers of present disclosure (right) showed strong bacterial sequestration ability that remain adhered even after washing with saline.

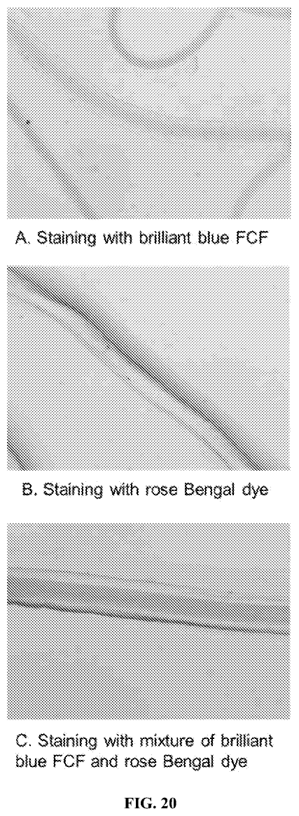

FIG. 20 shows microscopic images of proton-amide-fortified chitosan fibers depicting a clear distinction between protonated chitosan core (blue colored) and chitosan amide surface (pink colored) of fibers. The images were captured using differential uptake of dyes by the protonated chitosan and chitosan amide. The outer shell (chitosan amide) takes up rose Bengal (A), while the inner core of protonated chitosan takes up the brilliant blue FCF dye (B). When the samples were treated with both dyes simultaneously (C), the outer shell took up the rose Bengal dye (colored pink) and core took up the brilliant blue dye.

### DETAILED DESCRIPTION OF THE DISCLOSURE

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The use of the expression “at least” or “at least one” suggests the use of one or more elements or ingredients or quantities, as the use may be in the embodiment of the disclosure to achieve one or more of the desired objects or results. Throughout this specification, the word “comprise”, or variations such as “comprises” or “comprising” or “containing” or “has” or “having”, or “including but not limited to” wherever used, will be understood to imply the inclusion of a stated element, integer or step, or group of elements, integers or steps, but not the exclusion of any other element, integer or step, or group of elements, integers or steps.

Reference throughout this specification to “some embodiments”, “one embodiment” or “an embodiment” means that a particular feature, structure or characteristic described in connection with the embodiment may be included in at least one embodiment of the present disclosure. Thus, the appearances of the phrases “in some embodiments”, “in one embodiment” or “in an embodiment” in various places throughout this specification may not necessarily all refer to the same embodiment. It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

The term “about” as used herein encompasses variations of +/−10% and more preferably +/−5%, as such variations are appropriate for practicing the present invention.

The present disclosure provides a superabsorbent chitosan dressing comprising proton-amide-fortified chitosan fibers. The term “proton-amide-fortified chitosan fibers” as used herein refers to chitosan fibers composed of a chitosan amide and an acid salt of chitosan. Unlike prior art where deprotonated/neutral chitosan fibers are first treated to provide protonated fibers of chitosan and these protonated fibers are thereafter processed to form a sheet/fabric of dressing, the present disclosure starts with a sheet or roll or fabric composed of neutral chitosan fibers which is thereafter treated in a continuous method as described herein to convert neutral chitosan fibers into proton-amide-fortified chitosan fibers composed of a chitosan amide and an acid salt of chitosan. The treatment is provided in such a way that the core of an individual chitosan fiber is composed of an acid salt of chitosan, while the surface of the individual fiber is composed of a chitosan amide. See FIG. 1, lower panel.

This treated sheet/roll/fabric is thereafter simply cut in a desired size and shape to provide a dressing. The inventors have observed that the superabsorbent chitosan dressing prepared in this manner has a substantially higher fluid absorbency and retention capacity, superior dry and wet tensile strength and exhibits a superior cohesive gel formation compared to prior art dressings where unprotonated/neutral individual chitosan fibers are first treated to provide individual protonated fibers of chitosan which are then processed to form a dressing.

Further, the continuous method described here to prepare dressings from sheets/rolls/fabric of unprotonated/neutral chitosan takes substantially less amount of time, is inexpensive, simple, and can produce dressings at a large scale in very less amount of time compared to prior art methods.

In some embodiments, the present disclosure provides a chitosan-based superabsorbent dressing that has no crosslinking agent and a rapid continuous method of producing it. The dressing of the present disclosure is made up of non-woven chitosan fabric that is post-treated, unlike the prior art where the fibers are first treated and thereafter added with a crosslinking agent to provide dressing. The continuous method described herein is a simple and cost-effective method through which the chitosan dressing can be fortified/modified/protonated rapidly to impart the properties like wound exudate absorption and locking, hemostatic ability, and superior wet strength or cohesion during the removal.

The exudate locking ability, also referred to as fluid retention capacity in the present disclosure, means the percentage of total absorbed fluids that is held inside the fibers. This is an important property with respect to wound dressings as the fluid absorbed outside the fibers comes out when the dressing is compressed and require frequent dressing changes. The neutral chitosan fibers or simple protonated chitosan dressings of prior art also absorb the exudates, but do not hold the absorbed exudate when compressed. Hence the dressings which not only absorb the exudates but also retain the absorbed exudates throughout the application duration are desired.

The method for preparing the chitosan dressing of the present disclosure comprises treating sheet/roll/fabric of neutral chitosan fibers with a treatment solution comprising an acid, alcohol, and optionally a surfactant to produce proton-amide-fortified chitosan fiber wound dressing in a continuous manner. The acid helps in protonating the chitosan fibers, which helps in rapid absorption of wound exudates. However, simple protonated fibers tend to disintegrate upon fluid absorption. The cohesion strength of the dressing is another important parameter for an effective use of the dressing. The cohesion strength of the present dressing is substantially high. It is hypothesized that the cohesive strength has been imparted due to the fortification step through the drying of protonated chitosan fibers at temperatures above 50° C., preferably at or above 80° C., after the acid treatment of neutral chitosan sheet/roll/fabric. It is desirable that a dressing has optimum cohesive strength until the complete duration of the intended use and the dressing should be able to be removed in one piece after the intended use. The inventors found that significantly higher cohesive strength was imparted to dressings by transient exposure to temperatures above 50° C., preferably at or above 60° C., preferably at or above 70° C., or preferably at or above 80° C., which was not achieved with exposure below 50° C. temperatures. Relatively shorter duration of heat treatment employed in the present disclosure to achieve the cohesive strength compared to prior art comes from the fact that the amount of acid, alcohol and water in the treatment solution content is just right to help in the formation of chitosan-amide on the surface of the protonated fibers. The dressing of the present disclosure forms a cohesive gel after absorption of the fluid. To achieve this, the method of the present disclosure does not involve use of any chemical crosslinking agents as described in the prior art. Further, certain prior arts show that dressings with lower cohesion have higher absorption capacity and dressings with higher cohesion have lower absorption capacity, i.e., these prior art dressings have inverse relationship between cohesion and absorption. In contrast, dressings of the present disclosure show high cohesion as well as high absorption capacity.

FIG. 1 depicts a schematic representation showing the differences in structure, composition, fluid absorption behavior of neutral chitosan fibers, simple protonated chitosan fibers and proton-amide-fortified chitosan fibers of present disclosure. As shown, the neutral chitosan fibers contain chitosan molecules in its native deprotonated form, which is not water soluble. Hence these fibers do not absorb any fluids and do not swell in presence of aqueous solutions. The protonated chitosan fibers as reported in the prior art mostly contain acid-salt of chitosan which is water soluble. These fibers absorb the fluids excessively and swell enormously leading to quick disintegration of the dressings when exposed to excess amount of liquids. On the other hand, the proton-amide-fortified fibers of present disclosure have distinct protonated chitosan core and chitosan-amide surface. This results in superior absorption and fluid retention within the fibers without dissolution or disintegration in the presence of excess fluids. Therefore, the dressings prepared using superabsorbent fibers of present disclosure give a cohesive gel with superior wet strength on exudate absorption.

The relatively quicker process of the present disclosure to convert sheet/roll/fabric composed of neutral chitosan fibers into a proton-amide-fortified form of chitosan fibers (where the core of the fiber an acid salt of chitosan and the surface of the fiber is a chitosan amide) is advantageous in providing a dressing composed of chitosan fibers that are highly protonated inside and are fortified on the surface via the formation of chitosan amide. This property facilitates the formation of cohesive gel after exposure of dressing to the aqueous fluids or wound exudate.

The key parameters of method disclosed herein include composition of the treatment solution (mixture of acid, alcohol, water and surfactant), duration of treatment, ratio of neutral chitosan fibers and treatment solution, temperature and duration of drying step. Each of these steps are carefully monitored to consistently produce proton-amide-fortified chitosan dressings in the present disclosure.

The duration of treatment of sheet/roll/fabric of neutral chitosan fibers to provide protonated chitosan dressings is shorter for the present method compared to the time required for prior art methods. The shorter duration exposure at temperatures above 60° C. is important in ensuring that only the surface of fibers is fortified as chitosan-amide and core of the fiber remains as acid salt of chitosan. This method of the present disclosure provides a variation in the charge distribution on the surface and that of the interior of the fibers. If the dressing is entirely made up of protonated chitosan fibers, the dressing tends to dissolve completely thereby negatively affecting its cohesive strength (FIG. 1, middle panel). While, if the dressing is completely made up of chitosan-amide fibers, it does not provide any exudate absorption and gelling ability. Therefore, a controlled fortification/protonation of the neutral chitosan fibers is needed to impart excellent absorption and cohesive strength to the dressing when it comes in contact with wound exudates. The method of the present disclosure provides controlled protonation and amide formation in sheet/roll/fabric of chitosan fibers so that chitosan dressings with desired properties are obtained.

As discussed above, in prior methods, neutral chitosan fibers (not a fabric/roll/sheet) are treated with acid and optionally heated to convert the fibers into protonated form. In these methods, chitosan fibers are treated with acid for longer duration (e.g., a few minutes to even up to a few hours) because of which neutral chitosan is completely converted into the acid salt of chitosan. In the present method, a neutral chitosan fabric/roll/sheet is dipped into a treatment solution comprising an acid followed by removal of the excess treatment solution by squeezing and retaining just enough solution in the fabric/roll/sheet to cause controlled protonation of the fibers. It is followed by a transient exposure of the treated fabric/sheet/roll to elevated temperatures above 60 C. Under these processing conditions, neutral chitosan is not fully converted into the acid salt of chitosan; instead, interior part of the chitosan fiber forms an acid salt of chitosan whereas the surface part of the chitosan fiber forms a chitosan amide due to transient exposure to high temperature, which are referred as proton-amide fortified chitosan fibers in the present disclosure (FIG. 1, C). That is, the processing conditions of the present disclosure provide a proton-amide-fortified chitosan fabric/roll/sheet where individual fibers of chitosan are composed of an acid salt of chitosan (interior part/core of the fiber) and an amide of chitosan (exterior part/shell of the fiber). The relative proportion of the acid salt and the amide in individual fibers can vary; however, it is hypothesized that this combination of an acid salt in the interior part of the fiber and an amide on the outer part of the fiber provides superior fluid absorption, retention, tensile strength, and cohesion to the dressings of the present disclosure.

In some embodiments, provided herein is a superabsorbent dressing comprising a non-woven fabric composed of fibers comprising an acid salt of chitosan and a chitosan amide. In some embodiments, the chitosan amide constitutes about 5-20% by weight of the dressing, including values and ranges thereof. In some embodiments, the amount of chitosan amide in the dressing ranges from about 5-18%, 5-16%, 5-15%, 5-13%, 5-10%, 8-20%, 8-18%, 8-16%, 8-15%, 8-12%, 10-20%, 10-18%, 10-16%, 10-15%, 10-14%, 12-20%, 12-18%, 12-16%, 12-15%, 13-20%, 13-18%, 13-16%, 15-20%, or 15-17%, including values and ranges thereof. In some embodiments, the amount of chitosan amide in the dressing is about 5, 6, 7, 8, 9, 10, 11, 12, 13, 14, 15, 16, 17, 18, 19, or 20% by weight.

In some embodiments, the superabsorbent dressing comprises a chitosan amide selected from the group consisting of chitosan acetamide, chitosan succinimide, chitosan glycolic acid amide, chitosan lactamide, chitosan ascorbic acid amide, chitosan citric acid amide, chitosan tartaric acid amide, and a combination thereof. In an exemplary embodiment, the superabsorbent dressing comprises chitosan lactamide. One or more of these amides are present in the dressing in the amounts disclosed herein.

The acid salt of chitosan present in the superabsorbent dressing is a salt with an organic acid or a salt with a Lewis acid. In some embodiments, the superabsorbent dressing comprises a salt of chitosan with an organic acid, wherein the organic acid is selected from the group consisting of acetic acid, succinic acid, glycolic acid, lactic acid, ascorbic acid, citric acid, tartaric acid, and a combination thereof. In some embodiments, the superabsorbent dressing comprises a salt of chitosan with a Lewis acid, wherein the Lewis acid is selected from the group consisting of aluminium chloride, ferric chloride, aluminium lactate, and a combination thereof. In some embodiments, the superabsorbent dressing comprises a salt of chitosan with an organic acid or a salt with a Lewis acid.

In some embodiments, the acid salt of chitosan constitutes about 78-93%, 78-90%, 78-88%, 78-85%, 80-95%, 80-93%, 80-90%, 82-93%, 82-90%, 82-88%, 85-95%, 85-93%, 88-95%, or 88-93%, by weight of the superabsorbent dressing, including values and ranges thereof.

It would be understood by a person of ordinary skill in the art that the superabsorbent dressing comprises a chitosan amide and an acid salt of chitosan in any of the amounts disclosed herein. Further, any of the chitosan amides and any of the acid salts of chitosan disclosed herein form the main components of the superabsorbent dressing in any of the amounts disclosed herein.

In some embodiments, the superabsorbent dressing of the present disclosure comprises moisture in small amounts, such as, about 0.1-2%, 0.1-1.8%, 0.1-1.5%, 0.1-1.2%, 0.1-1%, 0.1-0.5%, 0.2-2%, 0.2-1.8%, 0.2-1.5%, 0.2-1%, 0.4-2%, 0.4-1.8%, 0.4-1.5%, 0.4-1%, 0.5-2%, 0.5-1.8%, 0.5-1.5%, 0.5-1%, 0.8-2%, 0.8-1.6%, 1-2%, or 1.5-2% by weight of the dressing.

In some embodiments, the superabsorbent dressing of the present disclosure comprises a surfactant. In some embodiments, the surfactant is a non-ionic surfactant. In some embodiments, the non-ionic surfactant is selected from the group consisting of polysorbate 20, polysorbate 80, lauryl glucoside, a poloxamer, and a combination thereof.

In an exemplary embodiment, the superabsorbent dressing of the present disclosure comprises a non-woven fabric composed of the fibers comprising chitosan lactate and chitosan lactamide. In another exemplary embodiment, the dressing of the present disclosure comprises a non-woven fabric composed of the fibers comprising chitosan acetamide and chitosan acetate. In some embodiments, the non-woven fabric further comprises a surfactant as described herein.

In some embodiments, provided herein is a superabsorbent dressing comprising a non-woven protonated chitosan fabric, sheet, or roll prepared by a method comprising: a) dipping a non-woven neutral chitosan fabric/sheet/roll in a treatment solution comprising an acid and an alcohol and optionally a surfactant to obtain a treated chitosan fabric/sheet/roll; b) removing excess amount of said treatment solution from the treated chitosan fabric/sheet/roll; and c) drying the treated chitosan fabric/sheet/roll obtained after removal of excess treatment solution at a temperature of about 60° C. or more to obtain the dressing. In some embodiments, in the step of removing excess amount of the treatment solution from the treated chitosan fabric/sheet/roll, the chitosan to treatment solution ratio is maintained at 1:2 to 1:10. That is, after removal of the excess treatment solution, in some embodiments, the ratio of chitosan to the treatment solution remaining in the chitosan fabric/sheet/roll is 1:2 to 1:10.

The treatment solution is prepared by adding an acid and optionally a surfactant and water to an alcohol.

In some embodiments, the acid employed in the treatment solution is selected from the group consisting of acetic acid, succinic acid, glycolic acid, lactic acid, ascorbic acid, citric acid, tartaric acid, and a combination thereof. In some embodiments, the present disclosure also contemplates using an inorganic acid for protonation of chitosan fabric/sheet/roll. The concentration of the acid in the treatment solution ranges from 0.001 g/mL to 0.1 g/mL, including values and range thereof, depending upon the type of the acid employed. In some embodiments, the concentration of the acid in the treatment solution is about 0.001-0.05, 0.001-0.04, or 0.005-0.05 g/mL.

In some embodiments, the alcohol employed in the treatment solution is selected from the group consisting of ethanol, isopropyl alcohol, a polyalcohol, and a combination thereof.

In some embodiments, the surfactant employed in the treatment solution is as described above.

The concentration of the surfactant can range from about 0.001% w/v to about 5% w/v.

In some embodiments, the treatment solution comprises water. The percentage of water in the treatment solution can range from about 0.1% to about 30% v/v.

Exemplary treatment solutions (TS1-8) are shown below:
TS1: Prepare 0.005 g/ml of lactic acid solution by mixing 27.7 g of 90% lactic acid in 5000 ml of ethanol. Surfactant tween 20 at 0.5% is added to this solution.
TS2: Prepare 0.01 g/ml of lactic acid solution by mixing 55.5 g of 90% lactic acid in 5000 ml of ethanol.
TS3: Prepare 0.02 g/ml of lactic acid solution by dissolving 111 g of 90% lactic acid in 5000 ml of isopropyl alcohol.
TS4: Prepare 0.04 g/ml of lactic acid solution by dissolving 222 g of 90% lactic acid in 5000 ml of isopropyl alcohol.
TS5: The treatment solution comprises a mixture of alcohol and water with acid and surfactant. Prepare by dissolving 222 g of 90% lactic acid in 5000 ml of solvent with ratio 90:10 of isopropyl alcohol:water.
TS6: Prepare 0.005 g/ml of acetic acid solution by adding 25 g of 5000 ml of ethanol.
TS7: Prepare 0.01 g/ml of acetic acid solution by dissolving 50 g of acetic acid in 5000 ml of isopropyl alcohol.
TS8: Prepare 0.02 g/ml of acetic acid solution by dissolving 100 g of acetic acid in 5000 ml of isopropyl alcohol. 25 g of tween 20 is added to this solution to enhance the wetting property of dressing.

The protonated non-woven superabsorbent chitosan dressings of the present disclosure show superior properties. In some embodiments, the superabsorbent dressing of the present disclosure shows a fluid absorbency of about 14, 20, 25, 30, 35, 40, 45, or 50 g/g or more such as about 14-100, 14-95, 14-90, 14-85, 14-80, 14-75, 14-70, 14-65, 14-60, 14-50, 20-100, 20-95, 20-90, 20-85, 20-80, 20-75, 20-70, 20-65, 20-60, 20-50, 25-100, 25-95, 25-90, 25-85, 25-80, 25-75, 25-70, 25-65, 25-60, 25-50, 30-100, 30-95, 30-90, 30-85, 30-80, 30-75, 30-70, 30-60, 30-50, 40-100, 40-90, 40-80, 40-70, 40-60, 50-100, 50-95, 50-90, 50-85, 50-80, 50-75, 55-100, 55-95, 55-90, 55-85, 55-80, 55-75, 60-100, 60-96, 60-90, 60-85, 60-80, 65-100, 65-95, 65-90, 65-85, 70-100, 70-95, 70-90 g/g.

In some embodiments, the superabsorbent dressing of the present disclosure shows a dry tensile strength of about 15 N or more, 20 N or more, 25 N or more, or 30 N or more such as about 15-45, 15-40, 15-35, 15-30, 15-25, 20-45, 20-40, 20-35, 20-30, 20-25, 22-35, 22-38, 22-35, 23-30, 23-28, 25-45, 25-40, 25-35, 25-30, 30-45, 30-40, 30-35, 32-45, 32-40, 32-38, 32-35, 33-40, or 33-38 N.

In some embodiments, the superabsorbent dressing of the present disclosure shows a wet tensile strength of about 1 N or more, 5 N or more or 10 N or more, such as about 1-20 N, 1-15 N, 1-10 N, 1-5 N, 5-20 N, 5-15 N, 5-10 N, 10-25 N, 10-20 N, or 10-15 N.

In some embodiments, the superabsorbent dressing of the present disclosure shows a fluid retention capacity of about 40% or more such as about 40-100%, 40-95%, 40-90%, 40-85%, 40-80%, 40-75%, 40-70%, 45-100%, 45-95%, 45-90%, 45-85%, 45-80%, 45-75%, 45-70%, 50-100%, 50-95%, 50-90%, 50-85%, 50-80%, 50-75%, 55-100%, 55-95%, 55-90%, 55-85%, 55-80%, 60-100%, 60-95%, 60-90%, 60-85%, 65-100%, 65-95%, 65-90%, 65-85%, 70-100%, 70-95%, or 70-90%.

In some embodiments, the superabsorbent chitosan dressing of the present disclosure shows affinity to blood cells, i.e., blood cells adhere to or are adsorbed on the chitosan fibers of the dressing. The inventors found that blood cells adhere to the chitosan dressings of the present disclosure where the fibers are composed of chitosan amide and an acid salt of chitosan but not to neutral/unprotonated dressings.

In some embodiments, the superabsorbent chitosan dressing sequesters bacteria. The term “sequester” as used herein means that bacteria are adsorbed on the surface of the dressing and are immobilized/trapped in the dressing.

In some embodiments, the superabsorbent dressing exhibits antibacterial activity. In some embodiments, the present dressing reduces number of viable bacterial cells by about 20-99%, 20-95%, 20-90%, 20-85%, 20-80%, 20-70%, 20-60%, 20-50%, 20-40%, 25-95%, 25-99%, 25-90%, 25-85%, 25-80%, 25-70%, 25-60%, 25-50%, 30-99%, 30-95%, 30-90%, 30-80%, 30-75%, 30-70%, 30-60%, 30-50%, 40-99%, 40-95%, 40-90%, 40-80%, 40-75%, 40-70%, 50-99%, 50-95%, 50-90%, 50-80%, or 50-70%, including values and ranges therebetween, after about 6, 8, 10, 12, 18, 24, 48, or 72 hours from the application of the dressing.

It would be clear to one of ordinary skill in the art that the superabsorbent dressings of the present disclosure can show any combination of the properties described herein.

In some embodiments, provided herein is a method for preparing a superabsorbent dressing comprising a non-woven protonated chitosan fabric/sheet, said method comprising: a) dipping a non-woven neutral chitosan fabric/sheet/roll in a treatment solution comprising an organic acid and an alcohol and optionally a surfactant to obtain a treated chitosan fabric/sheet/roll; b) removing excess amount of said treatment solution from the treated chitosan fabric/sheet/roll; and c) drying the treated chitosan fabric/sheet/roll obtained after removal of excess treatment solution at a temperature of about 80° C. or more to obtain the superabsorbent dressing, wherein the method is a continuous method. In some embodiments, in the step of removing excess amount of the treatment solution from the treated chitosan fabric/sheet/roll, the chitosan to treatment solution ratio is maintained at 1:2 to 1:10. That is, after removal of the excess treatment solution, in some embodiments, the ratio of chitosan to the treatment solution remaining in the chitosan fabric/sheet/roll is 1:2 to 1:10.

The term “continuous method” refers to a process in which the product (the protonated non-woven chitosan dressing) comes out without interruption and not in groups. The present method starts with a roll or a reel of sheet or fabric composed of neutral chitosan fibers which is unwound in a continuous controlled manner to pass the portions of the sheet/fabric through the treatment solution, followed by pressure rollers to remove excess treatment solution, followed by a heat treatment in a drying chamber. The dried product comprises protonated chitosan fibers. The dried product, in some embodiments, can be wound to form a roll/reel which is thereafter cut to form dressings of desired size and shape. In some embodiments, the dried product as it comes out of the drying chamber is cut to form dressings of desired size and shape. As one can see, the continuous method of the present disclosure render the process of making protonated chitosan dressings very simple, time and cost-effective and at the same time, provides dressings with superior absorbency, fluid retention capacity, dry and wet tensile strength and coherency for gel formation.

Continuous process has many advantages over the batch processes that were described in the prior art. The required set up is simple with dipping unit, squeezing unit, and drying chamber. Small, medium, and large-scale production is feasible with continuous process. Complete control over the modification of fabric/roll/sheet and uniform modification of the whole fabric/roll/sheet is possible. The parameters like contact time with a treatment solution, squeezing pressure, drying temperature and speed can be controlled to achieve desired properties of the dressing.

This is in contrast to a batch process where the entire roll/reel of fabric/sheet is dipped in reagents. In the batch process, the contact time with the reagents cannot be controlled. Uneven or excess uptake of reagents can lead to improper wetting or surface gelling which affects the fluid absorption (FIG. 13). As uniform modification is not feasible in batch process, fibers need to be modified first and the modified fibers are then used to make fabric/sheet which involves steps like fiber opening and blending, nonwoven carding, cross lapping, needle loom, and the like. All this involves a large production setup and small or medium scale production of modified chitosan dressing is not feasible using the batch process.

Furthermore, in the continuous method, multiple steps of modification and drying can be employed to add different properties to the chitosan dressing. For example, the method can also be used for carboxymethylation, thiolation, quaternization, catechol group addition or any other modification of chitosan that can be used in the wound care and done in a continuous manner. As an example, the modification to achieve carboxymethylation of chitosan dressing can involve reaction steps like treatment with sodium hydroxide followed by monochloro acetic acid; these steps can also be done in a continuous manner as described herein.

An exemplary set-up to perform the method of modifying chitosan in a continuous manner is shown in FIG. 2. As shown in this figure, there can be one chamber or multiple chambers to contain various treatment solutions. In some embodiments, some or each of these chambers is succeeded by pressure-rollers to remove excess solutions. In some embodiments, there is a single drying chamber at the end of the set-up. In some embodiments, there is more than one drying chamber placed suitably.

In some embodiments, the continuous method for preparing a superabsorbent dressing comprising a proton-amide-fortified chitosan fibers comprises: a) dipping a neutral chitosan fabric/sheet/roll in a treatment solution at a speed of 30-100 cm/min to obtain a treated chitosan fabric/sheet/roll; b) removing excess amount of said treatment solution from the treated chitosan fabric/sheet/roll by applying a squeeze pressure of about 0.1 to 2 kg/cm²; and c) drying the treated chitosan fabric/sheet/roll obtained after removal of excess treatment solution at a temperature of about 80-120° C. to obtain the superabsorbent dressing.

In some embodiments, the non-woven neutral chitosan fabric/sheet/roll is dipped in a treatment solution at a speed of 30-100, 30-90, 30-80, 30-75, 30-70, 30-60, 30-50, 30-40, 40-100, 40-90, 40-80, 40-75, 40-70, 40-60, 40-50, 50-100, 50-90, 50-80, 50-75, 50-70, 50-60, 60-100, 60-90, 60-80, 60-75, 60-70, 70-100, 70-90, 70-80, or 80-100 cm/min. In an exemplary embodiment, the non-woven neutral chitosan fabric/sheet/roll is dipped in a treatment solution at a speed of 35 cm/min. In some embodiments, excess amount of the treatment solution is removed from the treated chitosan fabric/roll/sheet by applying a squeeze pressure of about 0.1 to 2 kg/cm² such as a squeeze pressure of about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 kg/cm². In some embodiments, the squeeze pressure is about 0.5-1.5 or 0.5-1 kg/cm².

In some embodiments, the treated chitosan fabric/roll/sheet, after removal of excess treatment solution, is dried at a temperature of about 60-150, 60-145, 60-140, 60-135, 60-130, 60-125, 60-120, 60-115, 60-110, 60-105, 60-100, 60-90, 60-80, 70-150, 70-145, 70-140, 70-135, 70-130, 70-125, 70-120, 70-115, 70-110, 70-100, 70-90, 80-150, 80-145, 80-140, 80-135, 80-130, 80-125, 80-120, 80-110, 80-105, 80-100, 80-95, 80-90, 85-150, 85-145, 85-140, 85-130, 85-120, 85-110, 85-105, 85-100, 85-95, 90-150, 90-145, 90-140, 90-135, 90-130, 90-120, 90-110, 90-105, 90-100, 95-150, 95-145, 95-140, 95-135, 95-130, 95-120, 95-110, 95-105, 100-150, 100-145, 100-140, 100-135, 100-130, 100-120, 100-110, 110-150, 110-140, 110-130, 120-150, 120-145, 120-140, 120-130, 125-150, 125-140, or 130-150° C.

In some embodiments, the heat treatment for drying is carried out for a duration of about 6-20 minutes, such as about 6-18, 6-15, 6-12, 10-15, or 10-12 minutes, including values and ranges thereof.

In some embodiments, said step of drying is carried out in a drying chamber that is at least 1.5 m long.

In some embodiments, the continuous method is performed by employing a padding mangle machine.

The present disclosure also provides a system or a device for preparing the dressings of the present disclosure by employing the continuous method of the disclosure. In some embodiments, the system or device for preparing a dressing comprising a non-woven protonated chitosan fabric/sheet/roll in a continuous manner comprises: an unwinder; a dipping chamber; squeezer/padding means; a drive/belt; a drying chamber; and a winder. An exemplary set-up showing these components is depicted in FIG. 2. The neutral fabric/roll/sheet is mounted on to the unwinder. A compensator sensor cuts off the squeezer motor if there is excessive fabric/roll/sheet between the drying chamber and the squeezers. This prevents the fabric from dropping on to the ground. The drive speed in the squeezer/padding means controls the speed of the squeezers/pressure rollers. The direction can be forward or backward. The lever system is available to move the squeezing rolls. Pressure or the squeezing force can be set or adjusted using this lever. The fabric from the squeezers is dried in a drying chamber at about 80° C. or more. At the end of the drying treatment, the neutral chitosan fabric/sheet/roll is converted into the dressing comprising protonated chitosan fabric/sheet/roll. The protonated chitosan fabric/sheet/roll is rolled or z-folded or cut into smaller sheets to obtain dressings of desired size and shape.

It is to be understood that the foregoing descriptive matter is illustrative of the disclosure and not a limitation. While considerable emphasis has been placed herein on the particular features of this disclosure, it will be appreciated that various modifications can be made, and that many changes can be made in the preferred embodiments without departing from the principles of the disclosure. Those skilled in the art will recognize that the embodiments herein can be practiced with modification within the spirit and scope of the embodiments as described herein. Similarly, additional embodiments and features of the present disclosure will be apparent to one of ordinary skill in art based upon description provided herein.

Descriptions of well-known/conventional methods/steps and techniques are omitted so as to not unnecessarily obscure the embodiments herein. Further, the disclosure herein provides for examples illustrating the above-described embodiments, and in order to illustrate the embodiments of the present disclosure certain aspects have been employed. The examples used herein for such illustration are intended merely to facilitate an understanding of ways in which the embodiments herein may be practiced and to further enable those of skill in the art to practice the embodiments herein. Accordingly, the following examples should not be construed as limiting the scope of the embodiments herein.

### EXAMPLES

### Example 1: Neutral Chitosan Fabric Treatment Machine

The machine showed in FIG. 2 is a padding mangle type of machine. This machine is used for the treatment and drying of chitosan fabric in large scale production. The machine has the unwinder (101), fabric dipping (102) & squeezer/padding machine (103), drive/belt (104), drying chamber (105) and the winder (106) (FIG. 2). The fabric/raw material roll which needs to be processed is mounted on to the unwinder. A compensator sensor cuts off the squeezer motor if there is excessive fabric between the drying chamber and the squeezing rollers. This prevents the fabric from dropping on to the ground. The drive speed in the padding machine controls the speed of the squeezers. The direction can be forward or backward. The lever system is available to move the squeezing rolls. Pressure or the squeezing force can be set or adjusted using this knob. The fabric fortified or modified in the padding mangle machine needs to be dried properly before being rolled or z-folded or cut into smaller sheets.

### Example 2: Modification of Chitosan Fabric/Sheet Using Acetic Acid

This Example shows that by controlling the drive speed, squeezing pressure, and drying temperature, a protonated chitosan dressing with superior fluid absorption, fluid retention, tensile strength, and cohesive strength is obtained.

A roll of winded non-woven neutral chitosan fabric was taken. The neutral fabric was unwound and dipped in a treatment solution containing 2% w/v acetic acid and 0.5% tween 20 in isopropyl alcohol. The excess treatment solution was removed, and the treated fabric was dried. The drive speed (the speed at which the fabric was dipped in the treatment solution), the squeezing pressure to remove excess treatment solution, and the temperature for drying was varied. Tables 1-3 show the effect of these process parameters on properties of the dressing.

TABLE 1
Effect of squeezing pressure on the properties of the final product

| | | | | | | | Fluid uptake | |
|---|---|---|---|---|---|---|---|---|
| | Drive | Squeezing | | | Absorption | Fluid | time/wicking | |
| | speed | Pressure | Temp | Wetting | capacity | retention | property (5 cm | Cohesive |
| # | (cm/min) | (kg/cm²) | (° C.) | property | (g/g) | capacity (%) | dressing) | strength |
| PV101 | 30 | 0.1 | 80 | Irregular | 20 ± 0.8 (Surface | Fabric | More than | Fabric gels and |
| | | | | wetting | gelling preventing | dissolves | 120 secs | disperses |
| | | | | | further uptake) | | | |
| PV102 | 30 | 0.2 | 80 | Irregular | 19 ± 0.5 (Surface | Fabric | More than | Fabric gels and |
| | | | | wetting | gelling preventing | dissolves | 60 secs | disperses |
| | | | | | further uptake) | | | |
| PV103 | 30 | 0.5 | 80 | Wetting | 42 ± 0.9 | 57.8 ± 0.3% | Less than 5 sec | Remains intact |
| PV104 | 30 | 1 | 80 | Wetting | 9 ± 1.2 (Squeezed | 10.2 ± 0.6% | Less than 5 sec | Remains intact |
| | | | | | out reagents) | | | |
| PV105 | 30 | 2 | 80 | Wetting | 9 ± 0.7 (Squeezed | 7 ± 0.4% | Less than 5 sec | Remains intact |
| | | | | | out reagents) | | | |

Based on the above observations in Table 1, 0.5 kg/cm² of squeeze pressure is suitable for better absorbency, fluid retention capacity, fluid uptake and cohesive strength of the fabric. Lower squeeze pressures resulted in varying properties at the surface and the bulk. At 0.5 kg/cm² pressure, the retention of the treatment solution was optimum so that the absorptive properties of the dressing remained the same at the surface or in the bulk. As per weight calculation, the optimum weight ratio of chitosan to treatment solution was between 1:2 to 1:10, more specifically between 1:4 to 1:6, meaning for every gram of chitosan between 2 to 10 grams of treatment solution was optimum. As the pressure increased the treatment solution was squeezed out early and there was poor absorption of the fluids.

TABLE 2
Effect of drive speed on the properties of the final product

| | Drive | Squeezing | | | | Fluid | Fluid | |
|---|---|---|---|---|---|---|---|---|
| | speed | Pressure | Temp | Wetting | Absorption | retention | uptake time | |
| # | (cm/min) | (kg/cm²) | (° C.) | property | (g/g) | capacity (%) | (5 cm dressing) | Cohesive strength |
| PV201 | 30 | 0.5 | 80 | Wetting | 42 ± 0.9 | 57.8 ± 0.3% | Less than 5 secs | Remains intact |
| PV202 | 35 | 0.5 | 80 | Wetting | 51 ± 1.2 | 60.1 ± 0.3% | Less than 5 secs | Remains intact |
| PV203 | 40 | 0.5 | 80 | Wetting | 39 ± 0.6 | 49.3 ± 0.7% | Less than 30 secs | Remains intact |
| PV204 | 50 | 0.5 | 80 | Wetting | 25 ± 0.6 | 42.1 ± 0.9% | Less than 30 secs | Remains intact |
| PV205 | 70 | 0.5 | 80 | Wetting | 15 ± 0.5 | 39.6 ± 0.2% | No uptake | Remains intact (no |
| | | | | | | | | gelling property) |
| PV207 | 100 | 0.5 | 80 | Wetting | 12 ± 1 | 32.1 ± 0.8% | No uptake | Remains intact (no |
| | | | | | | | | gelling property) |
| PV208 | 120 | 0.5 | 80 | Wetting | 12 ± 0.9 | 12.8 ± 0.5% | No uptake | Remains intact (no |
| | | | | | | | | gelling property) |
| PV209 | 150 | 0.5 | 80 | No | 9 ± 1 | 5.1 ± 1.2% | No uptake | Remains intact (no |
| | | | | wetting | | | | gelling property) |

Based on the above experiments, a drive speed of 35 cm/min provided maximum absorbency, fluid retention capacity, fluid uptake and cohesive strength of the fabric. If the drive is slower than 35 cm/min, the fabric surface gels, and the uptake of fluids or absorbency is low. Whereas if the drive speed is faster than 35 cm/min, the fabric does not get enough time to get contact with reagent for the fortification and poor absorptive properties are observed (Table 2).

TABLE 3
Effect of temperature on the properties of the final product

| | Drive | Squeezing | | | | Fluid | Fluid | |
|---|---|---|---|---|---|---|---|---|
| | speed | Pressure | Temp | Wetting | Absorption | retention | uptake time | |
| # | (cm/min) | (kg/cm²) | (° C.) | property | (g/g) | capacity (%) | (5 cm dressing) | Cohesive strength |
| PV301 | 35 | 0.5 | 50 | Fabric | 19 ± 3 | Fabric | No fluid uptake | Remains intact |
| | | | | dissolves | (fabric gels) | dissolves | property | |
| PV302 | 35 | 0.5 | 60 | Fabric | 21 ± 2 | Fabric | No fluid uptake | Remains intact |
| | | | | dissolves | (fabric gel) | dissolves | property | |
| PV303 | 35 | 0.5 | 80 | Wetting | 51 ± 1.2 | 60.1 ± 0.3% | Less than 5 secs | Remains intact |
| PV304 | 35 | 0.5 | 100 | Wetting | 43 ± 1 | 43.5 ± 0.1% | Less than 5 secs | Remains intact |
| PV305 | 35 | 0.5 | 110 | Wetting | 35 ± 2 | 36.4 ± 0.6% | Less than 5 secs | Remains intact |
| PV306 | 35 | 0.5 | 125 | Wetting | 14 ± 1 | 27.6 ± 0.4% | No fluid uptake | Remains intact (no |
| | | | | | | | property | gelling property) |
| PV307 | 35 | 0.5 | 150 | Wetting | 12 ± 2 | 11.5 ± 0.2% | No fluid uptake | Remains intact (no |
| | | | | | | | property | gelling property) |

As can be seen from Table 3, the temperature of 80° C. to 110° C. provided maximum absorbency, fluid retention capacity, fluid uptake and cohesive strength of the fabric. Below this temperature, the drying of the fabric was incomplete and eventually fabric dissolved in the medium. And when the temperature was more than 80° C., the treatment solution dried out quickly and the absorptivity of the modified fabric was reduced. Highest absorbency of fluid was observed above 50 g/g when the drive speed was 35 cm/min, squeezing pressure was 0.5 kg/cm² and the drying temperature was 80° C. In methods where neutral chitosan fibers (not the sheet/roll/fabric of chitosan) are first protonated and then converted into a sheet/fabric, temperatures higher than 80-110° C. are employed to evaporate the unused treatment solvents. In contrast, in the present method, temperatures in the range of about 80-110° C. provide dressings with desired properties.

### Example 3: Preparation of Wound Dressing

Chitosan wound dressing is prepared in a continuous process where the fabric is fortified/modified/protonated using an acid and an alcohol. Surfactant was used to increase the wetting and penetration of the reagents into the fabric. The said treatment was carried out in a continuous manner where the final product comes out as a fortified/modified/protonated dressing. The acid can be acetic acid, succinic acid, glycolic acid, lactic acid, ascorbic acid, citric acid, tartaric acid or any organic or inorganic acid that can form soluble salts with chitosan and that can be taken up by chitosan fabric through the capillary action to attain positive charge on to the fibers. The surfactant can be used or not and can be anything belonging to the category of wetting agents. These are generally used to improve the wettability of the fabric and to fasten the uptake of acid by the chitosan fibers in the fabric. Alcohol can be ethanol, isopropyl alcohol or any other alcohol that can dissolve the acids.

Other than the above example depicting fortification/modification/protonation, the method can also be used for the following procedures like carboxymethylation, thiolation, quaternization, catechol group addition or any other modification of chitosan that can be used in the wound care and done in a continuous manner. The modification can be from 10% to an extent of 100% of the fabric. As an example, the modification to achieve carboxymethylation of chitosan dressing involves multiple reagents and can involve reaction steps like treatment with sodium hydroxide followed by monochloro acetic acid that can be done in a continuous manner.

The alcohol can be used in pure form or in combination with water. Examples include ethanol or isopropyl alcohol or a polyalcohol. The alcohol has been chosen as the solvent having a boiling point over 50° C.

The formulations used in this Example are given below:
Formulation A101: Prepared 0.005 g/ml of lactic acid solution by mixing 27.7 g of 90% lactic acid in 5000 ml of ethanol. 25 g of tween 20 added to this solution to enhance the wetting property of dressing. Chitosan fabric (degree of deacetylation (DDA)≥90% and dtex 1.6 to 2.0) of quantity 1000 g was treated with prepared solution at a drive speed of 35 cm/min, squeezing pressure of 0.5 kg/cm² and the drying temperature of 80° C.
Formulation A102: Prepared 0.01 g/ml of lactic acid solution by mixing 55.5 g of 90% lactic acid in 5000 ml of ethanol. 25 g of tween 20 added to this solution to enhance the wetting property of dressing and 1000 g fabric was treated with prepared solution at a drive speed of 35 cm/min, squeezing pressure of 0.5 kg/cm² and the drying temperature of 80° C.
Formulation A103: Prepared 0.02 g/ml of Lactic acid solution by dissolving 111 g of 90% lactic acid in 5000 ml of isopropyl alcohol. 25 g of tween 20 added to this solution to enhance the wetting property of dressing and 1000 g fabric was treated with prepared solution at a drive speed of 35 cm/min, squeezing pressure of 0.5 kg/cm² and the drying temperature of 80° C.
Formulation A104: Prepared 0.04 g/ml of lactic acid solution by dissolving 222 g of 90% lactic acid in 5000 ml of isopropyl alcohol. 25 g of tween 20 added to this solution to enhance the wetting property of dressing and 1000 g fabric was treated with prepared solution at a drive speed of 35 cm/min, squeezing pressure of 0.5 kg/cm² and the drying temperature of 80° C.
Formulation A201: The reagent was a mixture of alcohol and water with acid and surfactant. It was prepared by dissolving 222 g of 90% lactic acid in 5000 ml of solvent with ratio 90:10 of isopropyl alcohol:water. 25 g of tween 20 added to this solution to enhance the wetting property of dressing 1000 g fabric was treated with prepared solution at a drive speed of 35 cm/min, squeezing pressure of 0.5 kg/cm² and the drying temperature of 80° C. The chitosan fabric produced using this formulation has higher absorbency, better fluid wicking property, fluid retention capacity than the formulations where there is no water.
Formulation A301: The modification can be done using acetic acid in place of lactic acid. Prepared 0.005 g/ml of acetic acid solution by adding 25 g to 5000 ml of ethanol. 25 g of tween 20 added to this solution to enhance the wetting property of dressing and 1000 g fabric was treated with prepared solution at a drive speed of 35 cm/min, squeezing pressure of 0.5 kg/cm² and the drying temperature of 80° C.
Formulation A302: Prepared 0.01 g/ml of acetic acid solution by dissolving 50 g of acetic acid in 5000 ml of isopropyl alcohol. 25 g of tween 20 added to this solution to enhance the wetting property of dressing and the 1000 g fabric was treated with prepared solution at a drive speed of 35 cm/min, squeezing pressure of 0.5 kg/cm² and the drying temperature of 80° C.
Formulation A303: Prepared 0.02 g/ml of acetic acid solution by dissolving 100 g of acetic acid in 5000 ml of isopropyl alcohol. 25 g of tween 20 added to this solution to enhance the wetting property of dressing and 1000 g fabric was treated with prepared solution at a drive speed of 35 cm/min, squeezing pressure of 0.5 kg/cm² and the drying temperature of 80° C.
Formulation A304: Prepared 0.04 g/ml of acetic acid solution by dissolving 200 g of acetic acid in 5000 ml of solvent with ratio 90:10 of isopropyl alcohol:water. 25 g of tween 20 added to this solution to enhance the wetting property of dressing and 1000 g fabric was treated with prepared solution at a drive speed of 35 cm/min, squeezing pressure of 0.5 kg/cm² and the drying temperature of 80° C.
Formulation A401: Prepared 0.04 g/ml of citric acid solution by dissolving 200 g of citric acid in 5000 ml of solvent with ratio 90:10 of isopropyl alcohol:water. 25 g of tween 20 added to this solution to enhance the wetting property of dressing and 1000 g fabric was treated with prepared solution at a drive speed of 35 cm/min, squeezing pressure of 0.5 kg/cm² and the drying temperature of 80° C.
Formulation A501: Prepared 0.04 g/ml of ascorbic acid solution by dissolving 200 g of ascorbic acid in 5000 ml of solvent with ratio 90:10 of isopropyl alcohol:water. 25 g of tween 20 added to this solution to enhance the wetting property of dressing and 1000 g fabric was treated with prepared solution at a drive speed of 35 cm/min, squeezing pressure of 0.5 kg/cm² and the drying temperature of 80° C.
Formulation A601: Prepared 0.04 g/ml of glycolic acid solution by dissolving 200 g of glycolic acid in 5000 ml of solvent with ratio 90:10 of isopropyl alcohol:water. 25 g of tween 20 added to this solution to enhance the wetting property of dressing and 1000 g fabric was treated with prepared solution at a drive speed of 35 cm/min, squeezing pressure of 0.5 kg/cm² and the drying temperature of 80° C.
Formulation A701: Prepared 0.04 g/ml of succinic acid solution by dissolving 200 g of succinic acid in 5000 ml of solvent with ratio 90:10 of isopropyl alcohol:water. 25 g of tween 20 added to this solution to enhance the wetting property of dressing and 1000 g fabric was treated with prepared solution at a drive speed of 35 cm/min, squeezing pressure of 0.5 kg/cm² and the drying temperature of 80° C.
Formulation LA101: Prepared 0.01 g/ml of aluminium chloride solution by dissolving 50 g of aluminium chloride hexahydrate in 5000 ml of solvent with ratio 90:10 of isopropyl alcohol:water. 25 g of tween 20 added to this solution to enhance the wetting property of dressing and 1000 g fabric was treated with prepared solution at a drive speed of 35 cm/min, squeezing pressure of 0.5 kg/cm² and the drying temperature of 80° C.
Formulation LA201: Prepared 0.01 g/ml of ferric chloride solution by dissolving 50 g of ferric chloride hexahydrate in 5000 ml of solvent with ratio 90:10 of isopropyl alcohol:water. 25 g of tween 20 added to this solution to enhance the wetting property of dressing and 1000 g fabric was treated with prepared solution at a drive speed of 35 cm/min, squeezing pressure of 0.5 kg/cm² and the drying temperature of 80° C.
Formulation LA301: Prepared 0.01 g/ml of aluminium lactate solution by dissolving 50 g of aluminium lactate in 5000 ml of solvent with ratio 90:10 of isopropyl alcohol:water. 25 g of tween 20 added to this solution to enhance the wetting property of dressing and 1000 g fabric was treated with prepared solution at a drive speed of 35 cm/min, squeezing pressure of 0.5 kg/cm² and the drying temperature of 80° C.

The neutral/neutral chitosan fabric does not have the water or wound exudate absorptive property. However, when the chitosan fabric is protonated, it can absorb large amounts of liquids and converts itself into a gel form. After converting to the gel, the dressing takes up the contour of the wound and acts as a scaffold for the wound healing. The chitosan in protonated form is attracted to the blood, mucus layer or deeply injured layers of the skin where the internal layers of the skin are exposed, due to the negative charge on the blood and injured sites. During this process, the dressing can provide hemostasis and absorb exudates oozing from the wound and provides symptomatic pain relief at the wound site. It can be impermeable to the bacteria as the fabric is non-woven and chitosan has antimicrobial properties.

### Example 4: Conversion of Lactate to Lactamide in the Dressing

To test the conversion of chitosan lactate into chitosan lactamide, lactic acid content in the dressing was determined using a spectrophotometric method at different stages of treatment process.

Lactic acid was found to be lower in the final dressing when compared to the dressing loaded with lactic acid just after the squeezing step (Table 4). This indicates that some amount of lactic acid has been converted to lactamide which is also confirmed by the FTIR studies. Excess heat treatment of lactic acid and chitosan can result in lactamide formation. Lactamide formation results in the insoluble form of the chitosan whereas chitosan lactate is soluble in water. The method employs temperatures above 80° C. for roughly 10 min. During this treatment, the surface of the microfibers was converted to amide and the core remains as soluble lactate form. The formation of lactamide from lactate is confirmed in the FTIR by a peak at around 1580 cm⁻¹ (FIG. 3). As shown in FIG. 1, outer surface converts to insoluble form and the interior turns to soluble form with the treatment of the fabric as described in Example 2. The amide is also formed with other acids like succinic acid and glycolic acid and can be observed with the help of FTIR spectra depicted in FIG. 4.

TABLE 4
Lactate content in the dressings during
various phases of manufacturing

| | Lactic acid content | |
|---|---|---|
| | (mmol per gram | |
| Treatment step | chitosan) | Note |
| Just after the squeezing step | 3.34 ± 0.03 | |
| Drying step temperature | | |
| Room temperature | 3.34 ± 0.03 | No conversion |
| | | to lactamide |
| At 60-70° C. | 3.32 ± 0.03 | Very low levels |
| | | of lactamide |
| 80-100° C. | 2.46 ± 0.02 | Approx. 25% Lactic |
| | | acid converted |
| | | to lactamide |
| At 120° C. | 1.71 ± 0.02 | ~50% lactic acid |
| | | converted to lactamide |

### Example 5: Lactamide Content by Gravimetric Analysis

Chitosan when added to lactic acid during the treatment forms chitosan lactate. However, with the right process conditions the chitosan lactate will turn into chitosan lactamide. The chitosan lactamide is insoluble as the free amine groups were covalently bound to give rise to the amide groups. This chitosan lactamide is insoluble in water and the right amount of chitosan lactamide in the dressings gives the dressings good tensile strength and water locking ability. If chitosan lactate is not processed further or suboptimal temperatures were used it will not convert to lactamide (FIGS. 5 & 6). These dressings will tend to gel completely in presence of water (FIG. 7). When there is not enough chitosan lactamide on the surface of the fibers or is not completely crosslinked, the fibers tend to burst open in presence of water (FIG. 8). However, when there is optimum chitosan lactamide it gives the dressings desired properties (FIG. 9). But when all the lactate is converted to lactamide the water absorbing and locking abilities were lost (FIG. 10). To determine the extent of lactamide content in the dressings gravimetric method was applied. The principle here is that the chitosan lactate will dissolve in water but chitosan lactamide will not. The undissolved part is weighed and percentage of it to the total dressings was calculated. The results were reported in Table 5. The fabric treated at temperatures around 80° C. tend to possess 5-16% of the chitosan lactamide by weight and has all the optimal properties to be used as a wound dressing. The dressings were also imaged using scanning electron microscope (FIG. 11). These dressings when subjected to freeze drying after water contact were imaged using scanning electron microscope (FIG. 12). They have shown the presence of the insoluble surface layer that is burst opened mostly due to the freeze-drying process. From all the data it can be understood that the process makes lactamide and 5-16% of chitosan lactamide in the final dressings makes the dressings suitable for wound care with good coherence, retention capacity and absorption abilities.

TABLE 5
Lactamide content in the dressings prepared
following different temperatures

| | Chitosan lactamide as | |
|---|---|---|
| | insoluble matter per | Conversion percentage |
| Temperature (° C.) | gram chitosan (%) | of initial lactic acid (%) |
| Room temperature | 0 | 0 |
| 60 | 0.18 ± 0.01 | 0.59 ± 0.02 |
| 80 | 9.06 ± 2.12 | 30.13 ± 1.23 |
| 100 | 15.76 ± 1.27 | 37.86 ± 3.41 |
| 125 | 25.65 ± 3.327 | 52.42 ± 3.57 |

### Example 6: Absorbency of Water and Saline by the Fortified/Protonated Chitosan Fabric

The developed non-woven chitosan dressings were studied for the absorbency of water as well as exudate absorption. Due to the capillary action and gelling ability of the fortified/protonated chitosan, it absorbs huge amounts of liquid. Treating the fabric additionally with a surfactant dramatically improved the wettability and absorbency of the fabric. The protonation of the fabric further increased the absorptivity of the fluid in the fabric (Table 6).

The absorbency was determined using a 2×2 cm sample of fortified fabric. Weight of the cut unit was noted down which is the dry weight of the sample (Wd). Then it was placed in 50 ml demineralized water or saline for 1 hour. After that it was kept in a petri dish for some time to decant the excess water. Then the wet weight (Ww) was noted, and the absorbency was calculated using the following formula. The results are given in Table 6 & 7. The results indicate that the combination of process variables and formulations with optimum water content result in good absorptivity of the dressing calculates as: Absorbency=Ww/Wd.

TABLE 6
Absorbency of the fortified/protonated
non-woven chitosan dressing in water

| Variants | Wetting | Absorbency (g/g) |
|---|---|---|
| Inactive non-woven | No wetting | 14 ± 0.6 |
| chitosan dressing | | |
| A101 | Wetting | 72.4 ± 0.5 |
| A104 | Wetting | 93.3 ± 0.6 |
| A201 | Wetting | 80.9 ± 0.6 |
| A303 | Wetting | 64.5 ± 0.7 |

TABLE 7
Absorbency of the fortified/protonated
non-woven chitosan dressings in saline

| | | Absorbency | |
|---|---|---|---|
| | | Dried at room | |
| Variant | Acid type | temperature | Dried at 80° C. |
| Untreated | Untreated | 11.15 ± 0.47 | 11.13 ± 0.81 |
| A201 | Lactic acid | 19.33 ± 0.54 | 20.15 ± 1.43 |
| A401 | Citric acid | 10.65 ± 0.79 | 11.27 ± 0.45 |
| A501 | Ascorbic acid | 13.86 ± 0.76 | 14.69 ± 0.14 |
| A601 | Glycolic acid | 20.22 ± 0.49 | 20.77 ± 0.37 |
| A701 | Succinic acid | 19.13 ± 0.31 | 19.99 ± 0.31 |
| LA101 | Aluminum chloride | 19.70 ± 0.97 | 19.84 ± 0.57 |
| LA201 | Ferric chloride | 20.47 ± 0.14 | 21.59 ± 0.98 |
| LA301 | Aluminum lactate | 13.82 ± 1.13 | 15.79 ± 0.19 |

### Example 7: Tensile Strength of the Chitosan Wound Dressing

The cohesive strength and tensile strength of the fibers was tested with various combinations of the process variables. The following table shows the tensile strength and cohesive strength of the fabric fortified with various formulations (Table 8 & 9). The cohesive strength of the fabric was very good, and it can be peeled off in one piece without damaging the dressing.

The sample was cut into 25 mm width and approximately 100 to 125 mm long, such that the dressing length of the sample (sample between the grips) is not less than 50 mm. The sample was soaked in saline for 30 mins to determine the wet strength. The test was performed using a universal tensile tester. The test parameters include breaking point 95%, test speed 100 mm/min and sample width 25 mm.

TABLE 8
Tensile strength and cohesive strength of the chitosan dressing in water

| | Inactive | | | | |
|---|---|---|---|---|---|
| Parameter | fabric | A101 | A104 | A201 | A303 |
| Dry tensile strength (N) | 15.9 ± 0.6 | 33.1 ± 0.7 | 32.3 ± 1.2 | 33.4 ± 1.4 | 32.5 ± 2.1 |
| Wet tensile strength (N) | 7.5 ± 0.1 | 11.6 ± 2.1 | 10.8 ± 1.4 | 13.5 ± 1.7 | 13.0 ± 0.3 |
| Cohesive strength | Remains | Remains | Remains | Remains | Remains |
| during removal | intact | intact | intact | intact | intact |

TABLE 9
Tensile strength of the fortified/protonated non-woven chitosan
dressings dried at different temperatures in saline

| | | Tensile strength | | | |
|---|---|---|---|---|---|
| | | Air dried | | Dried at 80° C. | |
| Variant | Acid type | Dry | Wet | Dry | Wet |
| Untreated | Untreated | 17.14 ± 0.62 | 13.72 ±0.71 | 17.79 ± 1.24 | 14.6 ± 1.06 |
| A201 | Lactic acid | 27.08 ± 0.72 | 1.7 ± 0.68 | 27.75 ± 0.46 | 1.96 ± 0.47 |
| A401 | Citric acid | 24.57 ± 0.94 | 14.15 ± 0.37 | 23.62 ± 2.70 | 13.23 ± 1.36 |
| A501 | Ascorbic acid | 26.17 ± 0.58 | 7.96 ± 0.30 | 23.65 ± 3.28 | 7.65 ± 1.36 |
| A601 | Glycolic acid | 26.94 ± 0.25 | 2.06 ± 1.2 | 23.26 ± 2.74 | 1.42 ± 0.25 |
| A701 | Succinic acid | 26.73 ± 0.93 | 1.6 ± 0.46 | 26.96 ± 2.84 | 0.54 ± 0.11 |
| LA101 | Aluminum chloride | 26.20 ± 1.61 | 6.51 ± 0.75 | 26.49 ± 1.28 | 6.35 ± 0.97 |
| LA201 | Ferric chloride | 25.40 ± 0.71 | 6.16 ± 0.76 | 25.51 ± 2.7 | 6.35 ± 1.00 |
| LA301 | Aluminum lactate | 24.41 ± 0.94 | 9.64 ± 0.67 | 24.54 ± 2.78 | 8.37 ± 0.68 |

### Example 8: Cohesive Strength TEST

Cohesive strength test or integrity test was carried out by taking 5×5 cm pieces of the fortified chitosan dressing. Then they were placed in the flask containing solution A for 60 sec and swirled gently without any vortex. The specimens were removed using forceps and determined the cohesive strength visually (FIGS. 13 & 14). To prepare solution A, 8.298 g of sodium chloride and 0.368 g of calcium chloride dihydrate were dissolved in deionized water and made up the volume to 1 liter.

### Example 9: Fluid Retention Capacity of the Chitosan Wound Dressing

A 5 cm diameter size of dressing was taken and saturated with normal saline for 24 h. After that, the sample was removed from the saline and weighed (w1), the sample was placed on a flat surface and 950 g of weight (which is equal to a pressure of 35 mm Hg) was placed on the sample for 3 minutes. The weight was removed after 3 minutes and the sample was reweighed (w2). The following formula was used to calculate the fluid retention capacity. The results are shown in Table 10 & 11. 5 to 20% water added to the treatment solution improved the fluid retention capacity and cohesive behavior of the fabric (FIGS. 15 & 16).

Percentage retention=[W2/W1]×100

TABLE 10
Retention capacity of various formulations in water

| Formulation | Retention capacity % |
|---|---|
| A101 | 61.8 ± 0.6 |
| A104 | 73.0 ± 0.5 |
| A201 | 85.9 ± 0.3 |
| A202 | 79.8 ± 0.7 |
| A203 | 45 ± 0.5 |
| A303 | 57.8 ± 0.5 |

TABLE 11
Retention capacity of the fortified/protonated
non-woven chitosan dressings in saline

| | | Retention capacity (%) | |
|---|---|---|---|
| | | Dried at room | |
| Variant | Acid type | temperature | Dried at 80° C. |
| Untreated | Untreated | 32.63 ± 1.45 | 34.09 ± 0.96 |
| A201 | Lactic acid | 68.07 ± 3.42 | 74.86 ± 0.82 |
| A401 | Citric acid | 44.38 ± 1.26 | 50.17 ± 0.40 |
| A501 | Ascorbic acid | 45.71 ± 2.77 | 48.42 ± 1.76 |
| A601 | Glycolic acid | 64.38 ± 0.80 | 64.61 ± 3.30 |
| A701 | Succinic acid | 65.66 ± 1.81 | 71.09 ± 1.99 |
| LA101 | Aluminum chloride | 46.55 ± 0.69 | 54.49 ± 0.77 |
| LA201 | Ferric chloride | 47.95 ± 1.36 | 55.59 ± 0.40 |
| LA301 | Aluminum lactate | 43.26 ± 0.39 | 47.79 ± 0.80 |

### Example 10: Hemostatic Ability of the Chitosan Wound Dressing

To measure the hemostatic ability of the dressing, 5×5 cm fortified chitosan dressing was placed in a beaker and 1 ml of citrated whole blood was added on the dressing using a micropipette. It was incubated at 37° C. for 5 min. Then 100 ml of deionized water was added to the beaker to hemolyze the unclotted RBCs. Clear solution indicates clotting, while colored solution due to unclotted blood leaking out of the sample indicates hemolysis and therefore improper clotting (FIG. 17).

### Example 11: Adhesion and Aggregation of Blood Cells by Proton-Amide-Fortified Chitosan Dressings

To measure the affinity of the chitosan wound dressing to the blood cells microscopic images after interaction of blood with the fibers have been taken. The blood cells adhere to the treated fibers but not to the untreated fibers. The cells were adhered very strongly that the washing with saline could not detach the cells from the fibers (FIG. 18).

### Example 12: Bacterial Sequestration of the Chitosan Wound Dressing

To measure the bacterial sequestration ability of the chitosan wound dressing microscopic images after interaction of the bacteria with the fibers have been taken. The bacterial cells adhere to the treated fibers but not to the untreated fibers. The cells were adhered very strongly that washing with saline could not detach the cells from the fibers (FIG. 19).

### Example 13: Antibacterial Effect of the Chitosan Wound Dressing

1 ml of the inoculum (approximately 2×10⁷ CFU/ml in PBS) was added to the dressing measuring 5×5 cm. The inoculated dressings were incubated at 37° C. in a sealed container for 0 minutes, and 24 hours, then transferred to 9 ml of neutralizing broth to neutralize any residual antimicrobial activity. It was vigorously vortex mixed to remove any viable organisms from the dressings. From this 0.1 ml was added to 9.9 ml of PBS and the number of viable organisms present was determined using a standard surface counting technique. The time-kill kinetics were calculated up to a 24-hour period.

TABLE 12
Log reduction efficiency of treated chitosan wound dressings

| Name of the | Initial | Final | Log | % |
|---|---|---|---|---|
| organism | CFU/ml | CFU/ml | reduction | reduction |
| S. aureus | 2 × 10⁷ | 102 | 5.24 | 99.999 |
| | | 110 | | |
| | | 126 | | |
| | | Avg - 112.6 | | |
| P. aeruginosa | 2 × 10⁷ | 121 | 5.24 | 99.999 |
| | | 117 | | |
| | | 109 | | |
| | | Avg - 115.6 | | |

### Example 14: Differential Color Uptake by the Core and Shell of the Fibers

To visualize the formation of distinct protonated chitosan core and chitosan-amide surface in same fibers, a counter staining method was used (FIG. 20). When the samples were stained with an acid dye Brilliant Blue FCF, only the core of fiber was stained blue and distinct unstained border is clearly visible (FIG. 20, A). Since brilliant blue is an acid dye, this indicated that the core of the fibers is more acidic due to presence of protonated chitosan.

Then the samples were stained with an anionic dye, Rose Bengal (FIG. 20, B). it only stained the surface of the fibers. Finally, the samples were stained with both dyes simultaneously, and as expected the core of fiber was stained blue and surface of fibers was stained pink (FIG. 20, C). This clearly indicated that the surface and core of the proton-amide-fortified chitosan fibers have distinct composition.

1. A superabsorbent chitosan dressing comprising chitosan fibers composed of chitosan amide and an acid salt of chitosan.

2. The superabsorbent chitosan dressing of claim 1, wherein the chitosan amide constitutes about 5-20% by weight of the dressing.

3. The superabsorbent chitosan dressing of claim 1, wherein the acid salt of chitosan constitutes about 78-93% by weight of the dressing.

4. The superabsorbent chitosan dressing of claim 1, wherein the chitosan amide is selected from the group consisting of chitosan acetamide, chitosan succinamide, chitosan glycolic acid amide, chitosan lactamide, chitosan ascorbic acid amide, chitosan citric acid amide, chitosan tartaric acid amide, and a combination thereof.

5. The superabsorbent chitosan dressing of claim 1, wherein the acid salt of chitosan is a salt of chitosan with an organic acid or a Lewis acid.

6. The superabsorbent chitosan dressing of claim 5, wherein the organic acid is selected from the group consisting of acetic acid, succinic acid, glycolic acid, lactic acid, ascorbic acid, citric acid, tartaric acid, and a combination thereof.

7. The superabsorbent chitosan dressing of claim 5, wherein the Lewis acid is selected from the group consisting of aluminium chloride, ferric chloride, aluminium lactate, and a combination thereof.

8. The superabsorbent chitosan dressing of claim 1, comprising a surfactant.

9. The superabsorbent chitosan dressing of claim 8, wherein the surfactant is a non-ionic surfactant selected from the group consisting of polysorbate 20, polysorbate 80, lauryl glucoside, a poloxamer, or a combination thereof.

10. A method for preparing the superabsorbent dressing of claim 1, comprising:
a. dipping a neutral chitosan fabric/sheet/roll in a treatment solution comprising an acid and an alcohol and optionally a surfactant to obtain a treated chitosan fabric/sheet/roll;
b. removing excess amount of said treatment solution from the treated chitosan fabric/sheet/roll; and
c. drying the treated chitosan fabric/sheet/roll obtained after removal of excess treatment solution at a temperature of about 60° C. or more to obtain the superabsorbent dressing.

11. (canceled)

12. The method of claim 10, wherein the acid is selected from the group consisting of acetic acid, succinic acid, glycolic acid, lactic acid, ascorbic acid, citric acid, tartaric acid, aluminium chloride, ferric chloride, aluminium lactate, and a combination thereof.

13. The method of claim 10, wherein the alcohol is selected from the group consisting of ethanol, isopropyl alcohol, a polyalcohol, and a combination thereof.

14. The method of claim 10, wherein the surfactant is a non-ionic surfactant selected from the group consisting of polysorbate 20, polysorbate 80, lauryl glucoside, a poloxamer, and a combination thereof.

15. The method of claim 10, wherein the treatment solution comprises water.

16. The superabsorbent dressing of claim 1, wherein the dressing shows a fluid absorbency of about 14 g/g or more, a dry tensile strength of about 15 N or more, a wet tensile strength of about 1 N or more, and/or a fluid retention capacity of about 40% or more.

17. (canceled)

18. (canceled)

19. (canceled)

20. The superabsorbent dressing of claim 1, wherein the dressing shows affinity to blood cells.

21. The superabsorbent dressing of claim 1, wherein the dressing sequesters bacteria.

22. The superabsorbent dressing of claim 1, wherein the dressing has bacterial reduction ability of at least 5 log base 10 or 99.999%.

23. (canceled)

24. The method of claim 10, wherein the neutral chitosan fabric/sheet/roll is dipped in the treatment solution at a speed of 30-100 cm/min; excess amount of the treatment solution is removed from the treated chitosan fabric/sheet/roll by uniform squeezing with pressure rollers at a squeeze pressure of about 0.1 to 2 kg/cm²; and drying the treated chitosan fabric/sheet/roll at a temperature ranging from about 60-150° C. in a drying chamber that is at least 1.5 m long.

25. The method of claim 10, wherein the neutral chitosan fabric/sheet/roll is dipped in the treatment solution at a speed of 35 cm/min; excess amount of the treatment solution is removed from the treated chitosan fabric/sheet/roll at a squeeze pressure of 0.5 kg/cm² and drying at a temperature of 80° C.

26. (canceled)

27. (canceled)

28. (canceled)

29. A device for preparing a dressing comprising a non-woven protonated chitosan fabric/sheet in a continuous manner, said device comprising: an unwinder; a dipping chamber; squeezer/padding means; a drive/belt; a drying chamber; and a winder.
